(19) United States
(12) Patent Application Publication
(10) Pub. No.: US 2008/0020971 A1
(43) Pub. Date: Jan. 24, 2008

(54) Heptanoic acid derivatives as dipeptidyl peptidase inhibitors

(75) Inventors: Villoo Morawala Patell, Bangalore (IN); Akash Mathur, Bangalore (IN); Amitesh Suman, Bangalore (IN); V. Fredrick Robin Devadoss, Bangalore (IN)

Correspondence Address: HAMRE, SCHUMANN, MUELLER & LARSON, P.C., P.O. BOX 2902, MINNEAPOLIS, MN, 55402-0902, US

(73) Assignee: Avestha Gengraine Technologies Pvt. Ltd., Bangalore (IN)

(21) Appl. No.: 11/714,404

(22) Filed: Mar. 6, 2007

Related U.S. Application Data
(60) Provisional application No. 60/778,938, filed on Mar. 6, 2006.

(51) Int. Cl. A61K 31/19; A61K 31/44; A61K 38/28; A61P 1/00; A61P 25/00; A61P 35/00; A61P 5/00; A61P 9/00; C07C 53/00; C40B 20/00

(52) U.S. Cl. 514/3; 506/2; 514/356; 514/557; 562/606

(57) ABSTRACT

The present invention is directed to novel heptanoic acid derivatives which are inhibitors of the dipeptidyl peptidase-IV enzyme (“DPP-IV inhibitors”) and which are useful in the treatment or prevention of diseases in which the dipeptidyl peptidase-IV enzyme is involved, particularly in the treatment of type 2 diabetes and conditions that are associated with the same. In addition, the present invention provides pharmaceutical compositions useful in inhibiting DPP-IV enzyme, comprising a therapeutically effective amount of heptanoic acid derivatives. Moreover, the present invention provides a method of inhibiting DPP-IV comprising administering to a mammal in need of such treatment a therapeutically effective amount of a single or a combination of heptanoic acid derivatives of the invention. The invention further relates to the kits and other articles of manufacture for treating disease states associated with DPP-IV enzyme. The invention further relates to a method of identifying a compound that has dipeptidyl peptidase-IV enzyme inhibition activity, comprising following steps: 1. Define the residues of the active site of DPP-IV 2. Define the geometry and force field relationship of the residues identified above in (1) 3. Define the physical parameters of the active site identified in (I) 4. Validate the model based on mutational analysis and in-vitro inhibitor binding studies 5. Screen the library for scaffolds and small molecules that satisfy the model developed in (3) and validated in (4) above. 6. Dock each inhibitor identified in (5) above to the active site of DPP-IV defined in (I). 7. Minimize the energy of the inhibitor and DPP-IV complex using force fields used in (2) above. 8. Compare the energy of interaction of each inhibitor to that of known inhibitors. 9. Synthesize and validate in in-vitro assays

This application claims the benefit of U.S. Provisional Application No. 60/778,938 filed on Mar. 6, 2006.

The patent or application file contains at least one drawing executed in color. Copies of this patent or patent application publication with color drawing(s) will be provided by the Office upon request and payment of the necessary fee.

### FIELD OF INVENTION

The present invention is directed to novel heptanoic acid derivatives which are inhibitors of the dipeptidyl peptidase-IV enzyme (“DPP-IV inhibitors”) and which are useful in the treatment or prevention of diseases in which the dipeptidyl peptidase-IV enzyme is involved, particularly in the treatment of type 2 diabetes and conditions that are associated with the same. In addition, the present invention provides pharmaceutical compositions useful in inhibiting DPP-IV enzyme, comprising a therapeutically effective amount of heptanoic acid derivatives. Moreover, the present invention provides a method of inhibiting DPP-IV comprising administering to a mammal in need of such treatment a therapeutically effective amount of a single or a combination of heptanoic acid derivatives of the invention.

The invention further relates to the kits and other articles of manufacture for treating disease states associated with DPP-IV enzyme.

The invention further relates to a method of identifying a compound that has dipeptidyl peptidase-IV enzyme inhibition activity, comprising following steps:
1. Define the residues of the active site of DPP-IV
2. Define the geometry and force field relationship of the residues identified above in (I)
3. Define the physical parameters of the active site identified in (I)
4. Validate the model based on mutational analysis and in-vitro inhibitor binding studies
5. Screen the library for scaffolds and small molecules that satisfy the model developed in (3) and validated in (4) above.
6. Dock each inhibitor identified in (5) above to the active site of DPP-IV defined in (1).
7. Minimize the energy of the inhibitor and DPP-IV complex using force fields used in (2) above.
8. Compare the energy of interaction of each inhibitor to that of known inhibitors.
9. Synthesize and validate in in-vitro assays

### BACKGROUND OF THE INVENTION

Diabetes mellitus is characterized by metabolic defects in production and utilization of carbohydrates, resulting in elevated blood glucose or hyperglycemia due to the failure to maintain appropriate blood sugar levels. Research in the treatment of diabetes has centered on attempts to normalize fasting and postprandial blood glucose levels. Current treatments include administration of exogenous insulin, oral administration of drugs and dietary therapies and exercise regimens.

Two major forms of diabetes mellitus are recognized. Type 1 diabetes, or insulin-dependent diabetes, is the result of an absolute deficiency of insulin, the hormone which regulates carbohydrate utilization. In type 2 diabetes, or noninsulin dependent diabetes mellitus(NIDDM), patients often have plasma insulin levels that are the same or even elevated compared to nondiabetic subjects ; however, these patients have developed a resistance to the insulin stimulating effect on glucose and lipid metabolism in the main insulin-sensitive tissues, which are muscle, liver and adipose tissues, and the plasma insulin levels, while elevated, are insufficient to overcome the pronounced insulin resistance.

Persistent or uncontrolled hyperglycemia is associated with increased and premature morbidity and mortality. Often abnormal glucose homeostasis is associated both directly and indirectly with alterations of the lipid, lipoprotein and apolipoprotein metabolism and other metabolic and hemodynamic disease. Therefore patients with Type 2 diabetes mellitus are at especially increased risk of macrovascular and microvascular complications, including coronary heart disease, stroke, peripheral vascular disease, hypertension, nephropathy, neuropathy, and retinopathy. Therefore, therapeutic control of glucose homeostasis, lipid metabolism and hypertension are critically important in the clinical management and treatment of diabetes mellitus.

The available treatments for type 2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat.

Increasing the plasma level of insulin by administration of sulfonylureas (e.g. tolbutamide and glipizide) or meglitinide, which stimulate the pancreatic P-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur.

The biguanides increase insulin sensitivity resulting in some correction of hyperglycemia. However, the two biguanides, phenformin and metformin, can induce lactic acidosis and nausea/diarrhea. Metformin has fewer side effects than phenformin and is often prescribed for the treatment of Type 2 diabetes.

The glitazones (i.e. 5-benzylthiazolidine-2,4-diones) are a more recently described class of compounds with potential for ameliorating many symptoms of type 2 diabetes. These agents substantially increase insulin sensitivity in muscle, liver and adipose tissue in several animal models of type 2 diabetes resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type II diabetes are agonists of the alpha, gamma or delta subtype, or a combination of these, and in many cases are chemically different from the glitazones (i.e., they are not thiazolidinediones). Serious side effects (e.g. liver toxicity) have occurred with some of the PPAR agonists, such as troglitazone.

New biochemical approaches that have been recently introduced or are still under development include treatment with alpha-glucosidase inhibitors (e.g. acarbose) and protein tyrosinephosphatase-1B (PTP-1B) inhibitors.

Compounds that are inhibitors of the dipeptidyl peptidase-IV (“DP-IV” or “DPP-IV”) enzyme are also under investigation as drugs that may be useful in the treatment of diabetes, and particularly type 2 diabetes. See for example WO 97/40832, WO98/19998, U.S. Pat. No. 5,939,560, Bioorg. Med. Chem. Lett., 6(10), 1163-1166 (1996); and Bioorg. Med. Chem. Lett., 6 (22), 2745-2748 (1996). The usefulness of DP-IV inhibitors in the treatment of type 2 diabetes is based on the fact that DP-IV in vivo readily inactivates glucagon like peptide-1 (GLP-1) and gastric inhibitory peptide(GIP). GLP-I and GIP are incretins and are produced when food is consumed. The incretins stimulate production of insulin. Inhibition of DP-IV leads to decreased inactivation of the incretins, and this in turn results in increased effectiveness of the incretins in stimulating production of insulin by the pancreas. DP-IV inhibition therefore results in an increased level of serum insulin.

Advantageously, since the incretins are produced by the body only when food is consumed, DP-IV inhibition is not expected to increase the level of insulin at inappropriate times, such as between meals, which can lead to excessively low blood sugar (hypoglycemia). Inhibition of DP-IV is therefore expected to increase insulin without increasing the risk of hypoglycemia, which is a dangerous side effect associated with the use of insulin secretagogues. DP-IV inhibitors may also have other therapeutic utilities, as discussed herein. DP-IV inhibitors have not been studied extensively to date, especially for utilities other than diabetes. New compounds are needed so that improved DP-IV inhibitors can be found for the treatment of diabetes and potentially other diseases and conditions.

### DESCRIPTION OF THE PRIOR ART

WO 95/15309 discloses certain peptide derivatives which are inhibitors of DPP-IV and, therefore, are useful in treating a number of DPP-IV mediated processes.

Archives of Biochemistry and Biophysics, Vol. 323, No. 1, pgs. 148-154 (1995) discloses certain aminoacylpyrrolidine-2-nitriles which are useful as DPP-IV inhibitors.

WO 95/34538 discloses certain pyrrolidides, phosphonates, azetidines, peptides and azaprolines which inhibit DPP-IV and, therefore, are useful in treating conditions mediated by DPP-IV inhibition.

WO 91/16339 discloses certain tetrapeptide boronic acids, which are DPP-IV inhibitors useful in treating autoimmune diseases and conditions mediated by IL-2 suppression.

WO 93/08259 discloses certain polypeptide boronic acids, which are DPP-IV inhibitors useful in treating autoimmune diseases and conditions mediated by IL-2 suppression.

East German Patent 158109 discloses certain N-protected peptidyl-hydroxamic acids and nitrobenzoyloxamides which are useful as, inter alia, DPP-IV inhibitors.

WO 95/29691 discloses, inter alia, certain dipeptide proline phosphonates which are DPP-IV inhibitors useful in the treatment of immune system disorders.

East German Patent 296075 discloses certain amino acid amides, which inhibit DPP-IV.

Bioorganic and Medicinal Chemistry Letters, Vol. 6, No. 10, pgs. 1163-1166 (1996) discloses certain 2-cyanopyrrolidines, which are inhibitors of DPP-IV.

J. Med. Chem., Vol. 39, pgs. 2087-2094 (1996) discloses certain prolineboronic acid-containing dipeptides which are inhibitors of DPP-IV.

Bioorganic and Medicinal Chemistry Letters, Vol. 6, No. 22, pgs. 2745-2748 (1996) discloses certain 4-cyanothiazolidides which are inhibitors of DPP-IV.

Eur J. Med. Chem., Vol. 32, pgs. 301-309 (1997) discloses certain homologues and 3-substituted analogues of pyrrolidides and thiazolidides which inhibit DPP-IV.

### SUMMARY OF THE INVENTION

The present invention provides novel heptanoic acid derivatives. These compounds are potent and selective inhibitors of DPP-IV, and are effective in treating conditions that may be regulated or normalized via inhibition of DPP-IV enzyme. The invention also concerns pharmaceutical compositions comprising the compounds of the instant invention, a method of inhibiting DPP-IV comprising administering to a patient in need of such treatment a therapeutically effective amount thereof, the compounds for use as a pharmaceutical, and their use in a process for the preparation of a medicament for treating a condition which may be regulated or normalized via inhibition of DPP-IV enzyme.

The invention is also directed to kits and other articles of manufacture for treating disease states associated with DPP-IV.

The invention further provides an Insilico-method of screening compounds that have dipeptidyl peptidase-IV enzyme inhibition activity.

### DESCRIPTION OF TABLES AND FIGURES

Table 1: List of the side chains/ interacting residues of Dipeptidyl peptidase IV (DPP4) with Inhibitor

Table 2: Chemical/Physical Nature of DPP4 active site residue Table 3: Total energy before minimization and after minimization for interacting residues of Inu8_B chain and Ile-Por-Ile.

FIG. 1A model DPP4 inhibitor showing Cartesian coordinates and force field

FIG. 2: Total number of compounds with respect to different scaffolds

FIG. 3: Ball and stick model is inhibitor, which forms hydrogen bond with DPP4 active site residues. (GLU 205,SER 209 & HIS 126)

FIG. 4: Active-Site Residues of DPP-IV showing interaction with the docked compound 7-(2-benzyl-3-sulfanyl-propanoyl) amino heptonoic acid. Active-Site Residues are shown in Stick model and the docked compound in ball-and-stick model.

### DETAILED DESCRIPTION OF THE INVENTION

The instant invention relates to novel substituted hepatonoic acid derivatives of formula 1:

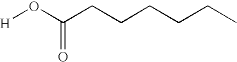

wherein

R1-R13 are each independently selected from the group consisting of

hydrogen,

halogen,

hydroxy,

cyano,

carboxy,

—SH

—PO₃H

C1-10 alkyl, wherein alkyl is unsubstituted or substituted with one to five substituents independently selected from halogen or hydroxy,

C 1-10 alkoxy, wherein alkoxy is unsubstituted or substituted with one to five substituents independently selected from halogen or hydroxy,

C 1-10 alkylthio, wherein alkylthio is unsubstituted or substituted with one to five substituents independently selected from halogen or hydroxy,

C2-10 alkenyl, wherein alkenyl is unsubstituted or substituted with one to five substituents independently selected from halogen or hydroxy,

(CH2) nCOOH,

(CH2)nCOOC₁₋₆alkyl,

(CH2)nCONR′R″, wherein R′ and R″ are independently selected from the group consisting of hydrogen, tetrazolyl, thiazolyl, (CH2) n-NRCOR7, (CH2) n-NR7Co2R6,(CH2) n-COR6, (CH2) n-C3-6 cycloalkyl, wherein cycloalkyl is unsubstituted or substituted with one to three substituents independently selected from halogen, hydroxy,C1-6 alkyl, andC1-6 alkoxy, wherein alkyl and alkoxy are unsubstituted or substituted with one to five halogens,

—(CH₂)ₙNR′R″

—(CH₂)ₙ—OCO NR′R″

—(CH₂)ₙ—SO₂ NR′R″

—(CH₂)ₙ—SO₂R′″

—(CH₂)ₙ—NR*SO₂R′″

—(CH₂)ₙ—NR*CONR′R″

—(CH₂)ₙ—NR*COR*

—(CH₂)ₙ—NR*CO₂R′″

—(CH₂)ₙ—COR′″

—(CH₂)ₙ—C₃₋₆ cyclo alkyl, wherein cycloalkyl is unsubstituted or substituted with one to three substituents independently selected from halogen, hydroxy,C1-6 alkyl, and C1-6 alkoxy, wherein alkyl and alkoxy are unsubstituted or substituted with one to five halogens,

—(CH2) n-aryl, wherein aryl is unsubstituted or substituted with one to five substituents independently selected from halogen, cyano, hydroxy,NR7S02R6,S02R6, C02H,C1-6 alkyloxycarbonyl, C1-6 alkyl, andC1-6 alkoxy, wherein alkyl and alkoxy are unsubstituted or substituted with one to five halogens,

—(CH2) n-heteroaryl, wherein heteroaryl is unsubstituted or substituted with one to three substituents independently selected from hydroxy, halogen, C1-6 alkyl, and C1-6 alkoxy, wherein alkyl and alkoxy are unsubstituted or substituted with one to five halogens, and

—(CH2) n-heterocyclyl, wherein heterocyclyl is unsubstituted or substituted with one to three substituents independently selected from oxo, hydroxy, halogen, C1-6 alkyl, andC1-6 alkoxy, wherein alkyl and alkoxy are unsubstituted or substituted with one to five halogens,

wherein any methylene (CH2) carbon atom inR1 or R2 is unsubstituted or substituted with one to two groups independently selected from halogen, hydroxy, andC1 4 alkyl unsubstituted or substituted with one to five halogens;

R′″ is independently selected from the group consisting of tetrazolyl, thiazolyl, (CH2)n-phenyl, (CH2)n-C3-6 cycloalkyl, and C1-6 aqlkyl, wherein alkyl is unsubstituted or substituted with one to five halogens and wherein phenyl and cycloalkyl are unsubstituted or substituted with one to five substituents independently selected from halogen, hydroxy, C1-6 alkyl, and C1-6 alkoxy, wherein alkyl and alkoxy are unsubstituted or substituted with one to five halogens, and wherein any methylene atom in R6 is unsubstituted or substituted with one or two groups independently selected from halogen, hydroxy, C1-4 alkyl, and C1-4 alkoxy,wherein alkyl and alkoxy are unsubstitued or substituted with one to five halogens.

Each R* is hydrogen or R′″

As used herein the following definitions are applicable.

### DEFINITIONS

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C.sub.3-10, the term alkyl also includes cycloalkyl groups, and combinations of linear or branched alkyl chains combined with cycloalkyl structures. When no number of carbon atoms is specified, C.sub.1-6 is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkoxy” refers to straight or branched chain alkoxides of the number of carbon atoms specified (e.g., C.sub.1-6 alkoxy), or any number within this range [i.e., methoxy(MeO—), ethoxy, isopropoxy, etc.].

The term “alkylthio” refers to straight or branched chain alkylsulfides of the number of carbon atoms specified (e.g., C.sub.1-6 alkylthio), or any number within this range [i.e., methylthio (MeS—), ethylthio, isopropylthio, etc.].

The term “alkylamino” refers to straight or branched alkylamines of the number of carbon atoms specified (e.g., C.sub.1-6 alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C.sub.1-6 alkylsulfonyl), or any number within this range [i.e., methylsulfonyl(MeSO.sub.2--), ethylsulfonyl, isopropylsulfonyl, etc.].

The term “alkyloxycarbonyl” refers to straight or branched chain esters of a carboxylic acid derivative of the present invention of the number of carbon atoms specified (e.g., C.sub.1-6 alkyloxycarbonyl), or any number within this range [i.e., methyloxycarbonyl(MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocycle” and “heterocyclyl” refer to saturated or unsaturated non-aromatic rings or ring systems containing at least one heteroatom selected from O, S and N, further including the oxidized forms of sulfur, namely SO and SO.sub.2. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like.

“Heteroaryl” means an aromatic or partially aromatic heterocycle that contains at least one ring heteroatom selected from O, S and N. Heteroaryls also include heteroaryls fused to other kinds of rings, such as aryls, cycloalkyls and heterocycles that are not aromatic. Examples of heteroaryl groups include pyrrolyl, isoxazolyl, isothiazolyl, pyrazolyl, pyridinyl, 2-oxo-(1H)-pyridinyl (2-hydroxy-pyridinyl), oxazolyl, 1,2,4-oxadiazolyl, 1,3,4-oxadiazolyl, thiadiazolyl, thiazolyl, imidazolyl, triazolyl, tetrazolyl, furyl, triazinyl, thienyl, pyrimidinyl, pyrazinyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, imidazo[1.2-.alpha.]pyridinyl, [1,2,4-triazolo][4,3-.alph-a.]pyridinyl, pyrazolo[1,5-.alpha.]pyridinyl, [1,2,4-triazolo][1,5-.alpha.-]pyridinyl, 2-oxo-1,3-benzoxazolyl, 4-oxo-3H-quinazolinyl, 3-oxo-[1,2,4]-triazolo[4,3-.alpha.]-2H-pyridinyl, 5-oxo-[1,2,4]-4H-oxadia-zolyl, 2-oxo-[1,3,4]-3H-oxadiazolyl, 2-oxo-1,3-dihydro-2H-imidazolyl, 3-oxo-2,4-dihydro-3H-1,2,4-triazolyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF.sub.30 and CF.sub.3CH.sub.2O).

Optical Isomers

The compounds of the present invention may contain one or more asymmetric centers and can thus occur as racemates and racemic mixtures, single enantiomers, diastereomeric mixtures and individual diastereomers. The compounds of the present invention have one asymmetric center at the carbon atom marked with an * in formula la. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

Salts

References to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methyinitrate, methylsulfate, mucate, napsylate, nitrate, N-1-22-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetate or maleate, can be employed. Included are those esters and acyl groups known in the art for modifying the solubility or hydrolysis characteristics for use as sustained-release or prodrug formulations.

Solvates, and in particular, the hydrates of the compounds of structural formula I are included in the present invention as well.

Metabolites—Prodrugs

Metabolites of the compounds of the invention that are therapeutically active and that are defined by Formula 1 are also within the scope of this invention. Prodrugs which are subsequently converted to a compund defined by formula 1 during or after administration are also within the scope of the invention.

Pharmaceutical Uses

Dipeptidyl peptidase-IV enzyme (DP-IV) is a cell surface protein that has been implicated in a wide range of biological functions. It has a broad tissue distribution (intestine, kidney, liver, pancreas, placenta, thymus, spleen, epithelial cells, vascular endothelium, lymphoid and myeloid cells, serum), and distinct tissue and cell-type expression levels. DP-IV is identical to the T cell activation marker CD26, and it can cleave a number of immunoregulatory, endocrine, and neurological peptides in vitro. This has suggested a potential role for this peptidase in a variety of disease processes in humans or other species.

Accordingly, the subject compounds are useful in a method for the prevention or treatment of the following diseases, disorders and conditions.

Type 2 Diabetes and Related Disorders: It is well established that the incretins GLP-1 and GIP are rapidly inactivated in vivo by DP-IV. Studies with DP-IV.sup.(−/−)-deficient mice and preliminary clinical trials indicate that DP-IV inhibition increases the steady state concentrations of GLP-1 and GIP, resulting in improved glucose tolerance. By analogy to GLP-1 and GIP, it is likely that other glucagon family peptides involved in glucose regulation are also inactivated by DP-IV (eg. PACAP). Inactivation of these peptides by DP-IV may also play a role in glucose homeostasis.

The DP-IV inhibitors of the present invention therefore have utility in the treatment of Type 2 diabetes and in the treatment and prevention of the numerous conditions that often accompany Type 2 diabetes, including metabolic syndrome X, reactive hypoglycemia, and diabetic dyslipidemia. Obesity, discussed below, is another condition that is often found with Type 2 diabetes that may respond to treatment with the compounds of this invention.

The following diseases, disorders and conditions are related to Type 2 diabetes, and therefore may be treated, controlled or in some cases prevented, by treatment with the compounds of this invention: (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) hypertension (24) Syndrome X, (25) ovarian hyperandrogenism (polycystic ovarian syndrome), and other disorders where insulin resistance is a component.

ObesitE: DP-IV inhibitors may be useful for the treatment of obesity. This is based on the observed inhibitory effects on food intake and gastric emptying of GLP-1 and GLP-2. Exogenous administration of GLP-1 in humans significantly decreases food intake and slows gastric emptying (Am. J. Physiol., 277: R910-R916 (1999)). ICV administration of GLP-1 in rats and mice also has profound effects on food intake (Nature Medicine, 2: 1254-1258 (1996)). This inhibition of feeding is not observed in GLP-IR.sup.(−/−) mice, indicating that these effects are mediated through brain GLP-1 receptors. By analogy to GLP-1, it is likely that GLP-2 is also regulated by DP-IV. ICV administration of GLP-2 also inhibits food intake, analogous to the effects observed with GLP-1 (Nature Medicine, 6: 802-807 (2000)). In addition, studies with DP-IV deficient mice suggest that these animals are resistant to diet-induced obesity and associated pathology (e.g. hyperinsulinonemia).

The subject compounds are further useful in a method for the prevention or treatment of the aforementioned diseases, disorders and conditions in combination with other agents.

Combination Therapy

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also includes therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula 1.

Examples of other active ingredients that may be administered in combination with a compound of Formula 1, and either administered separately or in the same pharmaceutical composition, include, but are not limited to:
(a) other dipeptidyl peptidase IV (DP-IV) inhibitors;
(b) insulin sensitizers including (i) PPAR.gamma. agonists such as the glitazones (e.g. troglitazone, pioglitazone, englitazone, MCC-555, rosiglitazone, and the like) and other PPAR ligands, including PPAR.alpha./.gamma. dual agonists, such as KRP-297, and PPAR.alpha. agonists such as fenofibric acid derivatives (gemfibrozil, clofibrate, fenofibrate and bezafibrate), (ii) biguanides such as metformin and phenformin, and (iii) protein tyrosine phosphatase-IB (PTP-IB) inhibitors;
(c) insulin or insulin mimetics;
(d) sulfonylureas and other insulin secretagogues, such as tolbutamide glyburide, glipizide, glimepiride, and meglitinides, such as repaglinide;
(e) alpha.-glucosidase inhibitors (such as acarbose and miglitol);
(f) glucagon receptor antagonists such as those disclosed in WO 98/04528, WO 99/01423, WO 00/39088, and WO 00/69810;
(g) GLP-1, GLP-1 mimetics, and GLP-I receptor agonists such as those disclosed in WO00/42026 and WO00/59887;
(h) GIP and GIP mimetics such as those disclosed in WO00/58360, and GIP receptor agonists;
(i) PACAP, PACAP mimetics, and PACAP receptor agonists such as those disclosed in WO 01/23420;
(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPAR.alpha. agonists such as fenofibric acid derivatives (gemfibrozil, clofibrate, fenofibrate and bezafibrate), (v) PPAR.alpha./.gamma. dual agonists, such as KPP-297, (vi) inhibitors of cholesterol absorption, such as beta-sitosterol and ezetimibe, (vii) acyl CoA:cholesterol acyltransferase inhibitors, such as avasimibe, and (viii) anti-oxidants, such as probucol;
(k) PPAR.delta. agonists, such as those disclosed in WO97/28149;
(l) antiobesity compounds such as fenfluramine, dexfenfluramine, phentermine, sibutramine, orlistat, neuropeptide Y.sub.1 or Y.sub.5 antagonists, CB1 receptor inverse agonists and antagonists, beta..sub.3 adrenergic receptor agonists, melanocortin-receptor agonists, in particular melanocortin-4 receptor agonists, ghrelin antagonists, and melanin-concentrating hormone (MCH) receptor antagonists;
(m)ileal bile acid transporter inhibitors;
(n) agents intended for use in inflammatory conditions such as aspirin, non-steroidal anti-inflammatory drugs, glucocorticoids, azulfidine, and selective cyclooxygenase-2 inhibitors; and
(o) antihypertensive agents such as ACE inhibitors (enalapril, lisinopril, captopril, quinapril, tandolapril), A-II receptor blockers (losartan, candesartan, irbesartan, valsartan, telmisartan, eprosartan), beta blockers and calcium channel blockers.

Antiobesity compounds that can be combined with compounds of structural formula I include fenfluramine, dexfenfluramine, phentermine, sibutramine, orlistat, neuropeptide Y.sub.1 or Y.sub.5 antagonists, cannabinoid CB1 receptor antagonists or inverse agonists, melanocortin receptor agonists, in particular, melanocortin-4 receptor agonists, ghrelin antagonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11: 1677-1692 (2001) and D. Spanswick and K. Lee, “Emerging antiobesity drugs,” Expert Opin. Emerging Drugs, 8: 217-237 (2003).

The above combinations include combinations of a compound of the present invention not only with one other active compound, but also with two or more other active compounds. Non-limiting examples include combinations of compounds having Formula I with two or more active compounds selected from biguanides, sulfonylureas, HMG-CoA reductase inhibitors, PPAR agonists, PTP-IB inhibitors, other DP-IV inhibitors, and antiobesity compounds.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

Administration and Dosage

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

Pharmaceutical Compositions

The pharmaceutical compositions for the administration of the compounds of this invention may conveniently be presented in dosage unit form and may be prepared by any of the methods well known in the art of pharmacy. All methods include the step of bringing the active ingredient into association with the carrier, which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouth washes and gargles.)

Kits Comprising DPP-IV Inhibitors

The invention is also directed to kits and other articles of manufacture for treating diseases associated with DPP-IV. It is noted that diseases are intended to cover all conditions for which the DPP-IV possesses activity that contributes to the pathology and/or symptomology of the condition.

In one embodiment, a kit is provided that comprises a composition comprising at least one DPP-IV inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one DPP-IV inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The pharmaceutical composition and method of the present invention may further comprise other therapeutically active compounds as noted herein which are usually applied in the treatment of the above mentioned pathological conditions.

In the treatment or prevention of conditions which require inhibition of dipeptidyl peptidase-IV enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about I mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in responsiveness of the mammal being treated for any of the indications with the compounds of the invention indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims, which follow, and that such claims be interpreted as broadly as is reasonable.

Assay: Measurement of Inhibition

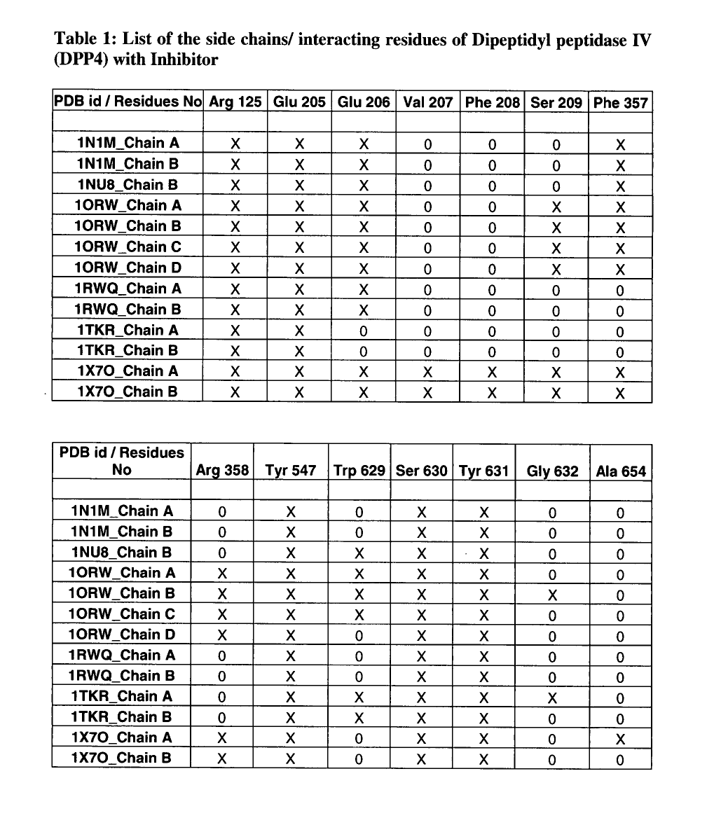

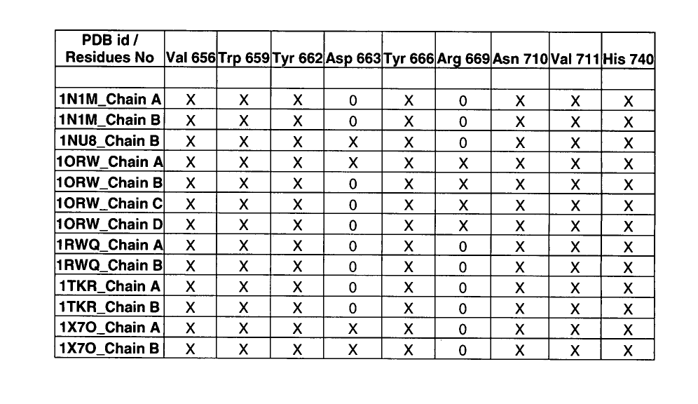

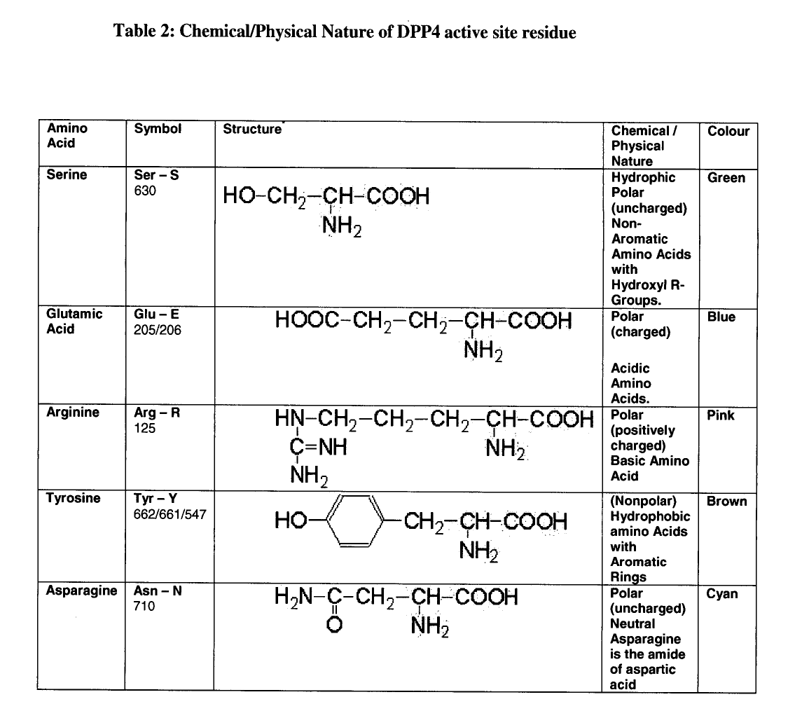

DPP-IV is an aminopeptidase that cleaves the sessile peptide bond two residues away from the amino terminus and requires the second residue to be Pro or Ala. For the assay synthetic substrate Gly-Pro-pNA was used that releases paranitroanilide after cleavage which is chromorphore absorbing at 405 nm (FIG. 1).

The DPPIV Protease Assay is a homogeneous, luminescent assay that measures dipeptidyl peptidase IV (DPPIV) activity. The DPPIV Assay provides a proluminescent DPPIV substrate, Gly-Pro-aminoluciferin, in a buffer system optimized for DPPIV and luciferase activities. The addition of a single DPPIV Reagent in an “add-mix-measure” format results in DPPIV cleavage of the substrate and generation of a “glow-type” luminescent signal produced by the luciferase reaction. In this homogeneous, coupled-enzyme format, the signal is proportional to the amount of DPP-IV activity present. The assay is designed for use with purified enzyme preparations. At 5nM enzyme and substrate concentrations used for the studies, the reaction was found to be linear up to 20 minutes.

The invention further relates to a method of identifying a compound that has dipeptidyl peptidase-IV enzyme inhibition activity, comprising following steps:

Step 1: Define the Residues of the Active Site of DPP-IV that are Critical to Inhibitor Interaction

DPP4 has been crystallized with different inhibitors. Select crystal structures were analyzed to identify the side chains of the protein that are within the distance of 2.5 to 5 angstroms from the inhibitor atoms. A list of the side chains/ interacting residues of Di peptidyl peptidase IV (DPP4) with Inhibitor is shown in table 1.

Step 2: Define the Geometry and Force Field Relationship of the Residues Identified Above in Step 1

A model DPP4 receptor was generated using the center of atoms as point charges and by defining the distance between charges and calculating the force field each atom would generate when immersed in water. For calculating the force field Amber Charges with Distance dependent dielectric constant was used. This model is the function of Cartesian coordinates and force field was used as the basis for the study. (FIG. 1)

Step 3: Define the Physical Parameters of the Active Site of DPP IV Enzyme Identified in Step 1

The model was further refined based on three physical parameters:
a.) Hydropathicity profile of the active site
b.) van der waals radii of the active site
c.) Space filling around the active site

The chemical and physical nature of DPP4 active site residues is shown in table 2.

Step 4: Validate the Model Based on Mutational Analysis and in-vitro Inhibitor Binding Studies

The model was validated by carrying out mutational analysis and in-vitro inhibitor binding studies.

Step 5: Screen the Library for Scaffolds and Small Molecules that Satisfy the Model Developed in Step 3

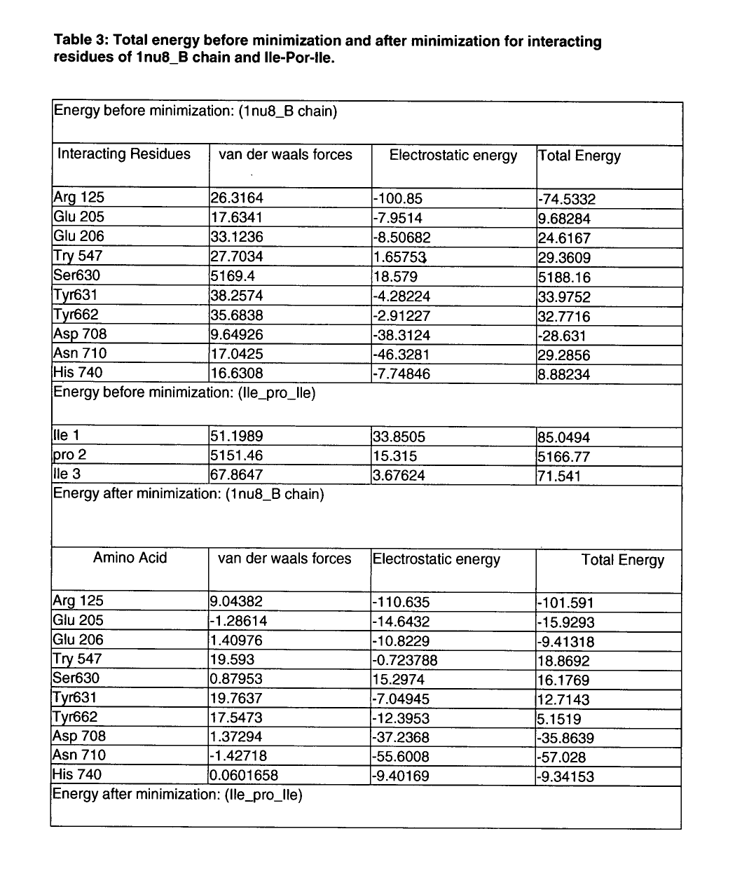

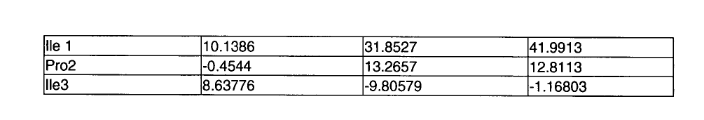

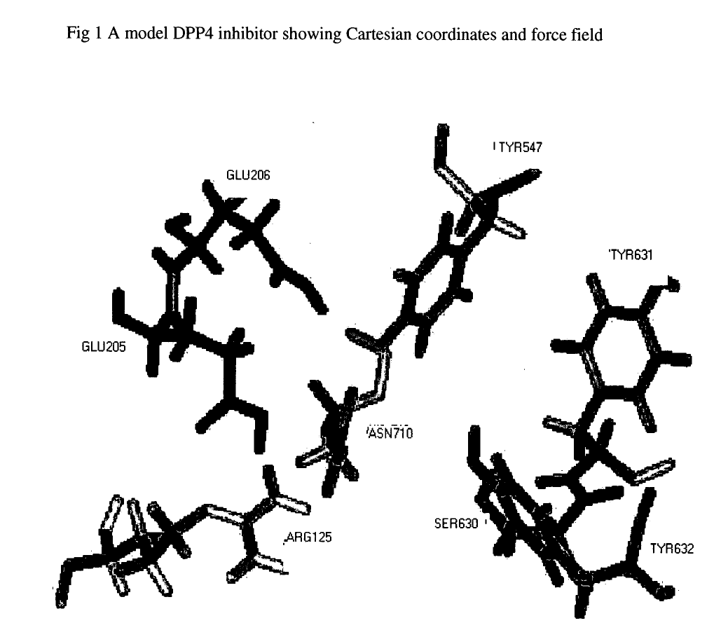

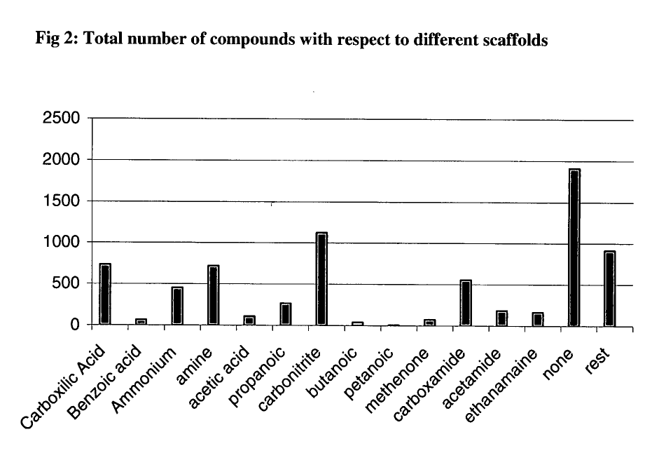

Based on the validated model developed above, a query for the library was generated using Cartesian coordinates, force fields, hydropathicity profiles, van der Waals radii and space available around each point charge representing the atom center. Each point in the query was weighted proportional to its score obtained in (1). Avesthagen chemical library was screened for molecules that satisfy this query. Initial screening resulted in identifying 5700 molecules of various scaffolds. Total number of compounds with respect to different scaffolds is shown in FIG. 2.

Step 6: Dock Each Inhibitor Identified in Step 5 Above to the Active Site of DPP-IV Defined in Step 1

Part 1: Molecular Mechanics Calculations:

a. Adding Hydrogen

X-ray crystallography cannot resolve hydrogen atoms in most protein crystals, so in most PDB files, hydrogen atoms are absent. So we have added hydrogen to fill all valences. Using (Insight II). Insight II is a comprehensive graphic molecular modeling program. In conjunction with molecular mechanics/dynamics programs, we can use the Insight II program to build and manipulate virtually any class of molecule or molecular system.

b. Assigning Potentials

Insightll cannot automatically fix potentials and charges. We assigned potentials using force field (FF) Discover (A molecular mechanics simulation environment offering energy minimization) module in insight II.

The Forcefield commands were used to assign partial charges and potential for energy calculations by the Insight II and Discover programs. We used these modules to identify unrecognized atoms and fix their potentials and charges. The Potentials command is used to check, fix (correct), or accept the potential function types of the atoms in a molecule. During assigning potential function atom types, Insight II first looks for matches in the currently assigned residue library. If a match is found, the residue library entry is used to assign the potential function atom types.

Part II: Sub-Set Formation for Receptor/Ligand:

We manually superimposed our ligand i.e. new_ile 97451(diprotein A) on the pre-existing synthetic ligand. Ligand was placed in the binding site to make interaction with the active site amino acids after removal of the ligand from the active site of DPPIV. A subset consisting of two central residues (GLU 205 ) was created and this will be treated as the binding site and the rest of the receptor molecule will be treated as BULK/RIGID. Bulk atoms are defined as atoms of the receptor that are not in the defined binding site. These atoms are held rigid during the course of the docking search.

Part III: Create Ligand/Receptor Assembly:

We created an assembly called (Lig_Host) using Insight II, which consist of ligand and receptor, wherein receptor was considered as object I and ligand as object 2.

Part IV: Energy Minimization:

Energy is a function of the degrees of freedom in a molecule (i.e. bonds, angles, and dihedrals). Conformational energy searching is used to find all of the energetically preferred conformations of a molecule (especially rotamers). Energy minimization process can precisely locate minimum energy Conformations .The goal of energy minimization is to find a route (consisting of variation of the intermolecular degrees of freedom) from an initial conformation to the nearest minimum energy conformation using the smallest number of calculations possible.

We have used two commonly available minimization algorithms (steepest and conjugate) module in insight II. Used for the first 10-100 steps of minimization. Table 3 gives the data of van der waals forces and electrostatic forces for interacting residues of Inu8_B chain and Ile-Pro-Iie, before energy minimization and after energy minimization.

Part V: Manual Docking:

In docking, the interaction energy is computed by summing the energy contributions between all atoms of the two molecules (receptor and ligand).

The objective of a docking type calculation is to evaluate the interaction energies of many orientations of one molecule relative to the other, while searching for the orientations that result in low interaction energies.

A critical step in the structure-based drug design process is the automatic docking of a flexible ligand to a protein active site. We used Affinity module for docking. The Affinity commands are located in the Docking module under the Affinity pull down. Affinity applies molecular mechanics in searching for and evaluating docked structures. In order to make the search fast enough for practical applications, the ligand/receptor system is partitioned into “bulk” and “movable” atoms. Bulk atoms are defined as atoms of the receptor that are not in the defined binding site. These atoms are held rigid during the course of the docking search. Movable atoms consist of atoms in the binding site of the receptor and ligand atoms. These atoms can move freely, except for binding site atoms close to bulk atoms. Affinity automatically docks ligands to receptors. For an assembly consisting of a ligand molecule and a receptor molecule (Lig_Host). Affinity uses the energy of the ligand/receptor complex to automatically find the best binding modes of the ligand to the receptor. This energy-driven method is especially useful in structure-based drug design where the experimentally determined structure of a protein-ligand complex is often unavailable. During the docking process, Affinity holds the ‘bulk’ of the receptor rigid, while the binding-site atoms and ligand atoms are movable.

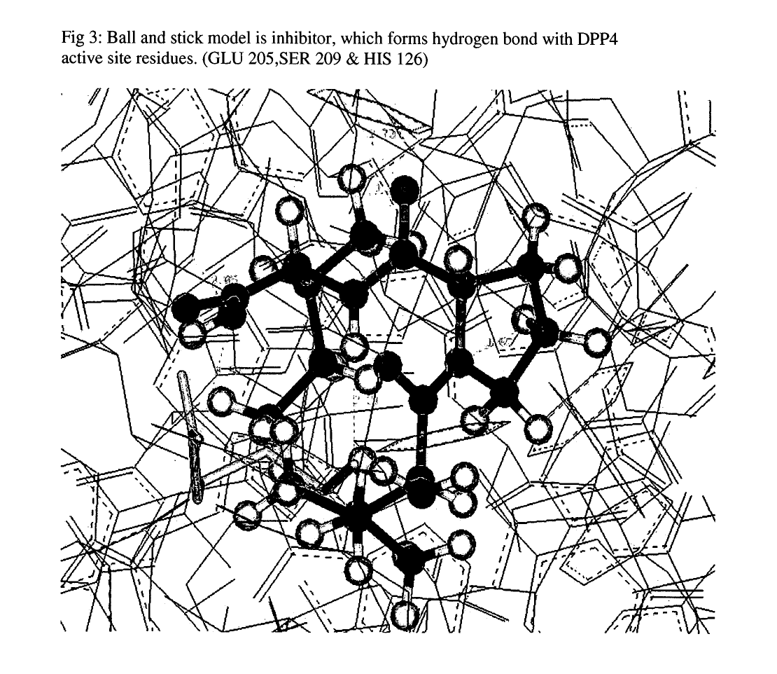

We moved the interacting molecules in real time on the screen while computing the interaction energy. While the energy expression is straightforward to compute, the computation time increases as the square of the number of interacting atoms, making the process too slow for many molecular systems. An energy grid approximating the larger of the two molecules can be pre-computed. Since calculating the energy between atoms of the moving molecule and the nearest grid points can then approximate the interaction energy. (FIG. 3)

Step 7: Minimize the Energy of the Inhibitor and DPP-IV Complex Using Force Fields Used in Step 2.

Step 8: Compare the Energy of Interaction of Each Inhibitor to that of Known Inhibitors.

### EXAMPLE

PDB INU8 chain B has been taken as the receptor and 7-(2-benzyl-3-sulfanyl-propanoyl) amino heptonoic acid as the ligand. The active site interacting residues of DPP-IV are Arg 125, Glu 205, Glu 206, Tyr 547, Tyr 631, Tyr 662 and Asn 710.

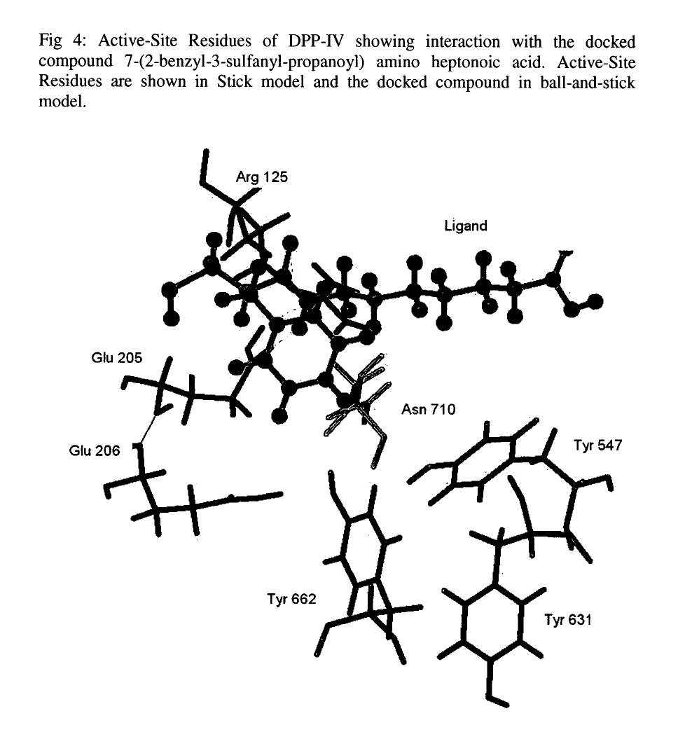

FIG. 4 shows the interaction of 7-(2-benzyl-3-sulfanyl-propanoyl) amino heptonoic acid with DPP-IV active site residues.

The energy has been calculated for the active site residues of the receptor as well as for the ligand. The energy has been compared with that of diprotin A—DPP-IV complex and 3-amino-4-phenyl betanoic acid—DPP-IV complex. The following analysis shows the energy of the three complexes.

Step 9: Synthesize and Validate in in-vitro Assays

Compounds under study would be synthesized and further in-vitro assays would be carried out to validate the DPP4 enzyme inhibition activity.

While the invention has been described and illustrated with reference to certain particular embodiments thereof, those skilled in the art will appreciate that various adaptations, changes, modifications, substitutions, deletions or additions of procedures and protocols may be made without departing from the scope of the invention. For example, effective dosages other than the particular dosage as set forth herein above may be applicable as a consequence of variations in responsiveness of the mammal being treated for any of the indications with the compounds of the invention indicated above. It is intended, therefore, that the invention be defined by the scope of the claims which follow and that such claims be interpreted as broadly as is reasonable.

1. A Compound of formula 1, wherein: 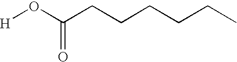
wherein
R1-R13 are each independently selected from the group consisting of
hydrogen,
halogen,
hydroxy,
cyano,
carboxy,
—SH
—PO₃H
C1-10 alkyl, wherein alkyl is unsubstituted or substituted with one to five substituents independently selected from halogen or hydroxy,
C 1-10 alkoxy, wherein alkoxy is unsubstituted or substituted with one to five substituents independently selected from halogen or hydroxy,
C 1-10 alkylthio, wherein alkylthio is unsubstituted or substituted with one to five substituents independently selected from halogen or hydroxy,
C2-10 alkenyl, wherein alkenyl is unsubstituted or substituted with one to five substituents independently selected from halogen or hydroxy,
(CH2)nCOOH,
(CH2)nCOOC₁₋₆alkyl,
(CH2)nCONR′R″, wherein R′ and R″ are independently selected from the group consisting of hydrogen, tetrazolyl, thiazolyl, (CH2) n-NRCOR7, (CH2) n-NR7Co2R6,(CH2) n-COR6, (CH2) n-C3-6 cycloalkyl, wherein cycloalkyl is unsubstituted or substituted with one to three substituents independently selected from halogen, hydroxy,C1-6 alkyl, andC1-6 alkoxy, wherein alkyl and alkoxy are unsubstituted or substituted with one to five halogens,
—(CH₂)ₙ-NR′R″
—(CH₂)ₙ—OCO NR′R″
(CH₂)ₙ—SO₂ NR′R″
—(CH₂)ₙ-SO₂R′″
—(CH₂)ₙ—NR*SO₂R′″
—(CH₂)ₙ—NR*CONR′R″
—(CH₂)ₙ—NR*COR*
—(CH₂)ₙ—NR*O₂R′″
—(CH₂)ₙ—COR′″
—(CH₂)ₙ—C₃₋₆ cyclo alkyl, wherein cycloalkyl is unsubstituted or substituted with one to three substituents independently selected from halogen, hydroxy,C1-6 alkyl, and C1-6 alkoxy, wherein alkyl and alkoxy are unsubstituted or substituted with one to five halogens,
—(CH2) n-aryl, wherein aryl is unsubstituted or substituted with one to five substituents independently selected from halogen, cyano, hydroxy,NR7S02R6,S02R6, C02H,C1-6 alkyloxycarbonyl, C1-6 alkyl, andC1-6 alkoxy, wherein alkyl and alkoxy are unsubstituted or substituted with one to five halogens,
—(CH2) n-heteroaryl, wherein heteroaryl is unsubstituted or substituted with one to three substituents independently selected from hydroxy, halogen, C1-6 alkyl, and C1-6 alkoxy, wherein alkyl and alkoxy are unsubstituted or substituted with one to five halogens, and
—(CH2) n-heterocyclyl, wherein heterocyclyl is unsubstituted or substituted with one to three substituents independently selected from oxo, hydroxy, halogen, C1-6 alkyl, andC1-6 alkoxy, wherein alkyl and alkoxy are unsubstituted or substituted with one to five halogens,
wherein any methylene (CH2) carbon atom inR1 or R2 is unsubstituted or substituted with one to two groups independently selected from halogen, hydroxy, andC1 4 alkyl unsubstituted or substituted with one to five halogens;
R′″ is independently selected from the group consisting of tetrazolyl, thiazolyl, (CH2)n-phenyl, (CH2)n-C3-6 cycloalkyl, and C1-6 aqlkyl, wherein alkyl is unsubstituted or substituted with one to five halogens and wherein phenyl and cycloalkyl are unsubstituted or substituted with one to five substituents independently selected from halogen, hydroxy, C1-6 alkyl, and C1-6 alkoxy, wherein alkyl and alkoxy are unsubstituted or substituted with one to five halogens, and wherein any methylene atom in R6 is unsubstituted or substituted with one or two groups independently selected from halogen, hydroxy, C1-4 alkyl, and C1-4 alkoxy,wherein alkyl and alkoxy are unsubstitued or substituted with one to five halogens.
Each R* is hydrogen or R′″
Or a pharmaceutically acceptable salt thereof.

2. A pharmaceutical composition, which comprises an inert carrier and a compound of claim 1.

3. A method for inhibition of dipeptidyl peptidase-IV enzyme activity in a mammal which comprises the administration to a mammalian patient in need thereof an effective amount of a compound of claim 1.

4. A method for treating, controlling, ameliorating or reducing the risk of diabetes comprising the administration to a mammalian patient in need thereof a therapeutically effective amount of a compound of claim 1.

5. A method for treating, controlling, ameliorating or reducing the risk of non-insulin dependent (Type 2) diabetes mellitus in a mammalian patient in need of such treatment which comprises administering to the patient a therapeutically effective amount of a compound of claim 1.

6. A method for treating, controlling, ameliorating or reducing the risk of hyperglycemia in a mammalian patient in need of such treatment which comprises administering to the patient a therapeutically effective amount of a compound of claim 1.

7. A method for treating, controlling, ameliorating or reducing the risk of obesity in a mammalian patient in need of such treatment which comprises administering to the patient a therapeutically effective amount of a compound of claim 1.

8. A method for treating, controlling, ameliorating or reducing the risk of insulin resistance in a mammalian patient in need of such treatment which comprises administering to the patient a therapeutically effective amount of a compound of claim 1.

9. A method for treating, controlling, ameliorating or reducing the risk of one or more lipid disorders selected from the group consisting of dyslipidemia, hyperlipidemia, hypertriglyceridemia, hypercholesterolemia, low HDL, and high LDL in a mammalian patient in need of such treatment which comprises administering to the patient a therapeutically effective amount of a compound of claim 1.

10. A method for treating, controlling or preventing atherosclerosis in a mammalian patient in need of such treatment which comprises administering to the patient a therapeutically effective amount of a compound of claim 1.

11. A method for treating, controlling, ameliorating or reducing the risk of one or more conditions selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) Syndrome X, (24) ovarian hyperandrogenism (polycystic ovarian syndrome), (25) hypertension and other disorders where insulin resistance is a component, in a mammalian patient in need thereof which comprises administering to the patient a therapeutically effective amount of a compound of claim 1.

12. A method for treating, controlling, ameliorating or reducing the risk of one or more conditions selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) irritable bowel syndrome, (15) inflammatory bowel disease, including Crohn's disease and ulcerative colitis, (16) other inflammatory conditions, (17) pancreatitis, (18) abdominal obesity, (19) neurodegenerative disease, (20) retinopathy, (21) nephropathy, (22) neuropathy, (23) Syndrome X, (24) ovarian hyperandrogenism (polycystic ovarian syndrome), (25) Type 2 diabetes, (26) growth hormone deficiency, (27) neutropenia, (28) neuronal disorders, (29) tumor metastasis, (30) benign prostatic hypertrophy, (32) gingivitis, (33) hypertension, (34) osteoporosis, and other conditions that may be affected by inhibition of DP-IV, in a mammalian patient in need thereof which comprisies administering to the patient a therapeutically effective amount of a first compound of claim 1, or a pharmaceutically acceptable salt thereof, and one or more other compounds selected from the group consisting of: (a) other dipeptidyl peptidase IV (DP-IV) inhibitors, (b) insulin sensitizers selected from the group consisting of (i) PPAR.gamma. agonists, other PPAR ligands, PPAR.alpha./.gamma. dual agonists, and PPAR.alpha. agonists, (ii) biguanides, and (iii) protein tyrosine phosphatase-IB (PTP-IB) inhibitors; (c) insulin or insulin mimetics; (d) sulfonylureas or other insulin secretagogues; (e) .alpha.-glucosidase inhibitors; (f) glucagon receptor agonists; (g) GLP-1, GLP-1 mimetics, and GLP-1 receptor agonists; (h) GIP, GIP mimetics, and GIP receptor agonists; (i) PACAP, PACAP mimetics, and PACAP receptor agonists; (1) cholesterol lowering agents selected from the group consisting of (i) HMG-CoA reductase inhibitors, (ii) sequestrants, (iii) nicotinyl alcohol, nicotinic acid or a salt thereof, (iv) PPAR.alpha. agonists, (v) PPAR.alpha./.gamma. dual agonists, (vi) inhibitors of cholesterol absorption, (vii) acyl CoA:cholesterol acyltransferase inhibitors, and (viii) anti-oxidants; k) PPAR.delta. agonists; (l) antiobesity compounds; (m) ileal bile acid transporter inhibitors; (n) antihypertensives; and (o) anti-inflammatory agents.

13. A method of identifying/ screening compounds that have dipeptidyl peptidase-IV enzyme inhibition activity, comprising following steps:
1. Define the residues of the active site of DPP-IV
2. Define the geometry and force field relationship of the residues identified above in (1)
3. Define the physical parameters of the active site identified in (1)
4. Validate the model based on mutational analysis and in-vitro inhibitor binding studies
5. Screen the library for scaffolds and small molecules that satisfy the model developed in (3) and validated in (4) above.
6. Dock each inhibitor identified in (5) above to the active site of DPP-IV defined in (1).
7. Minimize the energy of the inhibitor and DPP-IV complex using force fields used in (2) above.
8. Compare the energy of interaction of each inhibitor to that of known inhibitors.
